(19) United States
(12) Patent Application Publication
(10) Pub. No.: US 2004/0128703 A1
(43) Pub. Date: Jul. 1, 2004

(54) Methods and compositions for the generation of humanized mice

(76) Inventor: Hiroaki Shizuya, South Pasadena, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/659,034

(22) Filed: Sep. 9, 2003

Related U.S. Application Data
(60) Provisional application No. 60/409,631, filed on Sep. 9, 2002.

(51) Int. Cl. A01K 67/00

(52) U.S. Cl. 800/8; 800/21

(57) ABSTRACT

The invention provides methods and compositions for generating non-human transgenic animals that are humanized at one or more gene sequences. According to the methods of the invention, a DNA construct containing a human DNA sequence flanked by sequences from the non-human animal is generated by recombination in a bacterial cell, for example, in E. coli. The DNA construct that is produced can then be introduced into a non-human embryogenic stem cell where it can recombine with the genomic DNA of the non-human animal.

### RELATED APPLICATION DATA

This application claims priority under 35 USC 119(e) to U.S. Patent Application Serial No. 60/409,631 filed Sep. 9, 2002, herein incorporated by reference in its entirety.

### FIELD OF THE INVENTION

The invention relates to methods and compositions for the generation of humanized mice through homologous recombination using bacterial artificial chromosome.

### BACKGROUND INFORMATION

The DNA sequence of human genome has now been completed and the draft form of the DNA sequence of mouse genome has been reported. While sequencing efforts for several other higher eukaryotic organisms are in progress, the sequence information gathered will ultimately be converted into the genomic function information for understanding human diseases. The mouse has been an important experimental animal for studies in genetics and pathophysiology of a variety of human diseases. A wealth of information on mouse biochemistry, physiology and genetics is available to scientists. Most importantly, the ability of manipulating the mouse genome makes mouse unquestionably the most powerful animal tool for unraveling the pathogenesis of human diseases.

Several techniques currently exist for the generation of transgenic and other genetically modified mice. Transgenic mice (TM) can be generated by pronuclear injection and by viral transduction (C. Lois, E. J. Hong, S. Pease, E. J. Brown and D. Baltimore. (2002) “Germline Transmission and Tissue-Specific Expression of Transgenes Delivered by Lentiviral Vectors.” Science 295: 868). Unless such techniques are performed on a mouse background having the mouse gene corresponding to the transgene knocked out or otherwise disabled, the mouse generated will express both the mouse gene and the transgene product. Other techniques are being developed using recombination based approaches, but such approaches have limitations (Copeland et al. “Recombineering: A powerful new tool for mouse functional genomics. Nature Reviews—Genetics 2:769-779). These techniques universally rely on “partial” disruption or deletion of the endogenous gene and insertion of the human gene or genes (typically only a cDNA without introns) at random locations within the cell. Introns are usually not included, as the transferred human DNA is necessarily small in most cases. This means that some post transcriptional control mechanisms (e.g. that work during intron splicing) are lost. Splicing plays an important role in gene expression and transgenic mice made by cDNA lose this capacity.

Drugs are metabolized and transformed in the liver to more polar molecules for elimination. CYP450 enzymes are the primary drug metabolizing enzymes in the body. Three CYP450 subtypes are responsible for the majority of drug inactivation: CYP3A4, CYP2B6 and CYP2C9. Many drugs can induce the synthesis of CYP450 enzymes. The induction is an adaptive mechanism to protect the body from toxic chemicals, much like the immune system neutralizes foreign antigens in the body's attempt to fight pathogens. (Holmes VF. (1990) Rifampin-induced methadone withdrawal in AIDS. J Clin Psychopharmacol. 10:443-4.)

In addition to the CYP450 system, another site of drug-drug interactions is P-glycoprotein. This protein is encoded by multi-drug resistant (MDR1) gene and is a major efflux pump in the intestines involved in the excretion of many therapeutic agents. It is particularly effective in the elimination of anti-cancer drugs. Like the CYP450 system, P-glycoprotein is induced by different drugs, including rifampicin, SR12813, a selective human pregnane X receptor (PXR) agonist and Taxol (Synold et al. (2001) The orphan nuclear receptor SXR coordinately regulates drug metabolism and efflux. Nature Med. 7:584-590.). The increased expression of P-glycoprotein can greatly diminish the therapeutic levels of co-administered drugs. Importantly, many of the same compounds that induce CYP450, also induce P-glycoprotein.

The mechanism by which drugs induce CYP450 enzymes and P-glycoprotein involves the nuclear hormone receptor PXR. Studies by Xie et al. showed that the ability of drugs to induce CYP450 gene expression was abolished in mice with a PXR gene knock out. (Xie et al.

Humanized xenobiotic response in mice expressing nuclear receptor SXR. Nature 406:435-439; Xie W. and Evans R. (2001) Orphan nuclear receptors: The Exotics of Xenobiotics. J. Biol. Chem. 276:37739-37742.)

Comparison of the amino acid sequences of mouse and human PXR show only 72% identity in their ligand binding domains, which is relatively low for most nuclear receptors (Savas U. et al. (1999) Molecular mechanisms of cytochrome P-450 induction by xenobiotics: An expanded role for the nuclear hormone receptors. Mol Pharmacol. 56:851-857.). In contrast, the mouse and rat PXR binding domains are over 97% identical. The low sequence similarity between human and rodent PXRs appears to be responsible for the significant differences in ligand specificities of these receptors. A number of studies have now shown that the species differences in ligand binding pharmacology of PXR are responsible for the major differences in the ability of drugs to induce CYP450 expression in rodents and humans. In fact, Xie et al. (2000) showed, drugs such as rifampicin, clotrimazole, phenobarbital and 17β-estradiol, which stimulate CYP3A4 expression in human liver, had no effect on CYP3A expression in rat hepatocytes. However, by transfecting human PXR into the rat hepatocytes, these drugs were able to stimulate CYP3A expression. These authors went on to generate transgenic mice with the native PXR partially deleted and the human PXR gene targeted to the liver and showed that rifampicin and clotrimazole induced CYP3A expression in the mouse.

These species differences are a major problem in the drug development field. Since it is known that the CYP450 enzymes metabolize drugs and P-glycoprotein removes drugs from circulation, then any agent that stimulates the expression of these systems has the potential to cause drug-drug interactions to diminish the efficacy of co-administered drugs, and cause toxicity. This toxicity would only be apparent if several drugs were administered at the same time. Therefore, any new drug that is under pre-clinical development is usually tested for induction of the CYP450 system and MDR1.

However, rodent models are not good predictors of whether a drug can induce the CYP450 system and MDR1 in humans because of the ligand binding differences of rodent and human PXR. While one can test drugs for effects on human hepatocytes in vitro, in vitro systems are generally a poor substitution for in vivo testing for drug-drug interactions. Furthermore, because induction of the CYP450 and P-glycoprotein systems can have a significant effect on a drug's half-life, testing new drugs for efficacy, pharmacokinetics and toxicity in rodents may also not be a good predictor of actions in humans.

One approach to overcome this problem is to develop humanized mice that respond to inducers of the CYP450 system and MDR1 much like humans. Xie et al. (2000) generated mice in which the native PXR was deleted and the human receptor was expressed in the liver. However, these animals only partially recapitulated the human PXR system. First, the human PXR was targeted to the mouse liver but the human PXR also regulates CYP3A4 and MDR1 expression in the intestines, and the gastrointestinal tract is a major site of action of P-glycoprotein in eliminating drugs from the body.

Furthermore, PXR is expressed in tissues outside of the liver and intestines. Both human PXR and P-glycoprotein have been found to be co-expressed in kidney and placenta. This may suggest a role of PXR in renal drug metabolism and elimination. Furthermore, it may function to protect the placenta from xenobiotics. In addition, PXR and CYP450 are expressed in lungs where they are involved in the metabolism of air borne toxins. These potential interactions between PXR and CYP450 or P-glycoprotein are missed in the transgenic mouse created by Xie et al. In fact, all transgenic technology using cDNA do not allow for physiological expression of human genes in their normal tissue distribution.

Secondly, PXR does not work alone in regulating CYP450 expression. CAR is a major regulator of the expression of CYP2B genes and is responsible for mediating phenobarbital induction of CYP450 enzymes. Like PXR, there are significant variations in amino acid sequences and drug sensitivities of mouse and human CAR. The mouse and human CARs have only 72% amino acid sequence identity in their ligand binding domains. Molecular studies have shown that there is considerable cross talk between human PXR and CAR in regulating CYP450 genes.

In fact, while most studies have focused on the role of PXR in regulating the CYP450 and P-glycoprotein expression, it is likely that other factors are also involved in controlling the expression of these proteins in humans and contributing to drug-drug interactions. For example, PXR interacts with response elements in the CYP450 genes as a heterodimer with the retinoid X receptor (RXR). Retinoic acid, and the synthetic analogs, Rexinoids, which are ligands for the RXR receptor can activate human PXR/RXR dimers but not mouse or rat dimers (Jones, S. A. et al. (2000) The pregnane X receptor: a promiscuous xenobiotic receptor that has diverged during evolution. Mol. Endocrinol. 14: 27-39.). Thus, RXR may be a factor in the unique ligand specificity of the human PXR and therefore contribute to differences in drug-drug interactions found in humans that are not found in rodents. However, transgenic technologies using cDNA can not express multiple human genes in their natural location in mice so the coordinated regulation of the human CYP450 system and P-glycoprotein can't be reproduced with these approaches.

There therefore remains a need in the art for improved methods for the generation of genetically modified animals, useful for testing the effects of drugs as a predictor of the effects in humans.

### SUMMARY OF THE INVENTION

The present invention relates to methods for generating “humanized” animals having a human gene coding sequence in place of an orthologous endogenous animal gene coding sequence. In one embodiment, the human coding sequence also includes gene expression regulatory (control) regions. In another embodiment, the humanized animals have a human gene regulatory (control) region in place of an orthologous endogenous animal gene regulatory (control) region. Humanized mice are of particular utility to the pharmaceutical and biotechnology industry. Such humanized mice can be used, for example, to mimic human pharmacological and toxicological responses, create improved model systems for human disease and create improved models for drug responses to different human gene alleles.

According to the methods of the invention, a DNA construct containing a human DNA sequence flanked by sequences from the non-human animal is generated by recombination in a bacterial cell, preferably in E. coli. The DNA construct that is produced can then be introduced into a non-human embryogenic stem cell where it can recombine with the genomic DNA of the non-human animal. In another embodiment, the human DNA sequence is flanked by human regulatory sequences. In still another embodiment, a DNA construct containing a non-human animal DNA sequence flanked by human regulatory sequences is generated.

In one embodiment, the invention provides a method of generating a humanized animal involving recombining a first DNA construct with a second DNA construct. The first construct has a non-human animal DNA sequence contained therein and the second DNA construct has a human DNA sequence that is flanked by a first and a second non-human animal DNA sequence. Alternatively, the second construct has a human DNA sequence flanked by human regulatory sequences. In still another embodiment, the second has a non-human animal DNA sequence flanked by human sequences. In one embodiment, the sequences are derived from the same non-human animal as is desired to be constructed with the methods of the invention.

In one particular aspect, the first recombination step is carried out in a strain of E. coli that is deficient for sbcB, sbcC, recb, recC or recD activity and has a temperature sensitive mutation in recA. After the recombination step, a recombined third DNA construct is isolated, the construct having a human DNA sequence flanked by the first and second non-human animal DNA sequences; a human DNA sequence flanked by human sequences; or a non-human animal DNA sequence flanked by human sequences. The recombined construct is then introduced into a non-human embryogenic stem cell.

The invention also provides a DNA construct for performing homologous recombination within a cell, having a human DNA coding sequence having at least one intron and a selection marker gene contained within the at least one intron. The construct also has first and second non-human animal DNA sequences flanking the human DNA. The non-human animal flanking sequences are homologous to sequences in the genome of the non-human animal that flank a gene orthologous to the human DNA coding sequence. In one embodiment, recombination in an ES cell directs replacement of the non-human gene with its human orthologue. In another embodiment, the invention provides a DNA construct having a human DNA sequence flanked by human sequences. In still another embodiment, the invention provides a DNA construct having a non-human animal DNA sequence flanked by human sequences.

In another embodiment, the invention provides a method for generating a DNA construct for performing homologous recombination within a cell by recombination in a bacterial cell, preferably in E. coli. The DNA construct that is produced can then be introduced into a non-human embryogenic stem cell where it can recombine with the genomic DNA of the non-human animal.

In still another embodiment, the invention provides a humanized animal produced by the method of the invention. In another embodiment, the humanized animal is a mouse.

### BRIEF DESCRIPTION OF THE DRAWINGS

FIG. 1A is an illustration of a general procedure to generate fused DNA between mouse and human DNA. Two PCR products (pA and pB) are made; both are hybrid products between human and mouse DNA.

FIG. 1B is an illustration of PCR-1, carried out using primers p1 and p2. The resulting PCR products are hybrids between human and mouse DNA.

FIG. 1C shows the overlapping 20 bases between 3′ end of Product 1 and 5′ end of Product 2. Using primers p1 and p4, and the two products, PCR-5 generate ˜4 kb Product 5 that is a fused DNA at the overlapping region. Likewise, 4 kb Product 6 is generated as a fused DNA between Products 3 and 4.

FIG. 2 is an assembly of Products 5 and 6 and positive/negative markers by ligation. The resultant Product 7 is cloned into a BAC vector for subsequent generation of humanized mouse BAC.

FIG. 3 is general recombination between BAC-2 and linearized Product 7, as carried out in an E. coli strain.

FIG. 4 illustrates the recombination of linearized Product 9 with the orthologous mouse gene in the mouse genome by general recombination.

FIG. 5A is an illustration of the general procedure to generate fused DNA between mouse and human DNA where the desired regions flank the coding region of a gene to include regulator sequences both to the 5′ and 3′ of the gene. Two PCR products (pA and pB) are made; both are hybrid products between human and mouse DNA.

FIG. 5B is an illustration of PCR-1, carried out using primers p1 and p2. The resulting PCR products are hybrids between human and mouse DNA.

FIG. 5C shows the overlapping 20 bases between 3′ end of Product 1 and 5′ end of Product 2 from FIG. 5B. Using primers p1 and p4, and the two products, PCR-5 generate ˜4 kb Product 5 that is a fused DNA at the overlapping region. Likewise, ˜4 kb Product 6 is generated as a fused DNA between Products 3 and 4.

FIG. 6 is an assembly of Products 5 and 6 of FIG. 5C and positive/negative markers by ligation. The resultant Product 7 is cloned into a BAC vector for subsequent generation of humanized mouse BAC.

FIG. 7 is general recombination between BAC-2 and linearized Product 7 of FIG. 6, as carried out in an E. coli strain.

FIG. 8 illustrates the recombination of linearized Product 9 with the orthologous mouse gene in the mouse genome by general recombination.

FIG. 9 illustrates creation of the 5′ head chimera and the 3′ tail chimera in construction of a humanized PXR mouse.

FIG. 10 illustrates merging of the 5′ head chimera and the 3′ tail chimera of FIG. 9 and cloning into a pBAC vector.

FIG. 11 illustrates insertion of the teta gene into the Cla1 site of the pBAC vector of FIG. 10.

### DETAILED DESCRIPTION OF THE INVENTION

The invention provides an animal model of the human drug metabolism system. The invention utilizes bacterial artificial chromosomes (BAC) to generate mice expressing human PXR in its natural locations. As used herein, “natural location” is used to describe both the actual location of the gene coding sequence, e.g., on chromosome 16, and the orthologous endogenous characteristics of the gene. BAC allows for very long stretches of human DNA to be inserted into mice or other non-human animals. These stretches are much longer than used in standard cDNA transfer technologies to produce transgenic mice and, optionally, allow for tissue selective regulatory regions to be included along with the gene coding regions. As a consequence, the gene in question is expressed under proper control by the promoter of the deleted endogenous gene or if desired, under the control of the corresponding human regulatory region in its normal locations in the body at physiological levels rather than in every cell or in one site in the body due to standard gene targeting procedures. For example, previous studies (Nielsen L. et al. (1997) Human apolipoprotein B transgenic mice generated with 207- and 145 kb pair BAC. Evidence that distant 5′-element confers appropriate transgene expression in intestine. J. Biol. Chem. 272:29752-29758) showed that using standard transgenic procedures to express human ApoB gene in mice, the gene was expressed in liver, but not intestine of the mice. In contrast, using BAC, a 150-200 kb human ApoB gene was inserted in mice and expressed in both liver and intestine, thus recapitulating in the mouse the normal human expression. Further examples of the utility of the BAC system are described in Shizuya and Hosein-Mehr (Shizuya H. & Kouros-Mehr H. (2001) The development and application of the BAC cloning system. Keio J. Med. 50:26-30.) and Neuhausen (Neuhausen S. et al. (1994) A P1-based physical map of the region from D 17S776 to D 17S78 containing the breast cancer susceptibility gene BRAC 1. Hum Mol. Genet. 3:1919-1926.).

One advantage of the claimed invention is a large reduction in cost to pursue particular drug candidates because those candidates may be screened at an early stage of drug development. New technologies and tools to assist in making the decision as to which candidates to pursue are critical for pharmaceutical industry to save valuable resources in people and funds. In particular, initiating human trials based on poorly predictive efficacy and toxicology from animal trials are very costly and time consuming and may pose unnecessary risks to patients. Therefore, there is a great need for a reliable animal model for use in drug evaluation in the pre-clinical trials.

The BAC humanized transgenic mice prepared by the method of the invention provide the following advantages over prior methods: allow proper tissue specific expression, allow endogenous regulation of expression, provide physiological levels of expression, are precise regarding the site of integration, provide for removal of the endogenous coding region, provide for gene splicing and allow transgenes of about 1-350 kb, for example, greater than about 1 kb, 10 kb, 50 kb, 100 kb, 200 kb, 300 kb, 350 kb and the like, which is limited primarily by the size of the coding region and the size of the vector, e.g. BAC.

In one embodiment of the BAC system, very large genes (greater than 150 Kb, E.g. the Ig locus in humans is almost 970 Kb, too large for one BAC) can be assembled by sequentially replacing contiguous regions of orthologous very large genes by successive BAC transfers in F2 homozygotic animals. The present invention allows for creation of an animal with 150 Kb of the human gene, then creation of a subsequent animal with transfer of the next 150 Kb and so on.

The animal model of the invention can possess any of multiple combinations of inserted genes. In one embodiment, the animal has a human gene coding sequence in place of an orthologous endogenous animal gene coding sequence. In another embodiment, the human coding sequence also includes gene expression regulatory (control) regions, such that the animal possesses both human control and human coding regions for the orthologous gene. In another embodiment, the humanized animals have a human gene regulatory (control) region in place of an orthologous endogenous animal gene regulatory (control) region, but retain the endogenous coding region.

Additionally, BAC allows expression of multiple human genes in a rodent host. For example, one could potentially express human PXR, CAR and RXR as well as the target genes and the human promoters they regulate, all in the same animal. As such, the invention allows addition of multiple genes on a single BAC. As a consequence, gene networks could be inserted into BAC mice. Entire gene clusters or multiple gene pathways, such as human metabolic pathways, immunoglobulins, and the like either with or without their associated human regulatory sequences can be expressed in an animal host with multiple human genes. Insertion of gene networks or clusters with “normal” coordinated tissue and inducible expression may not be practicable with other transgenic technologies. For example, using the methods of the present invention, sequential genes could be added to an ES line that could be used to create a transgenic BAC animal, or transgenic animals could be made with ES lines containing one or more (but typically not all) of the desired genes and then cross bred with other transgenic BAC animals containing additional desired network or cluster genes.

Furthermore, the BAC system has flexibility. One can, through cross-breeding, add additional genes to the BAC mice. Thus, in mice, the basic foundation of the human system involved in induction of the CYP450 and MDR1 genes is produced and, as more is known about other elements that contribute to drug-drug interactions, genes for those elements could be added to the humanized mouse.

A humanized BAC mouse has a number of important uses for the pharmaceutical industry in drug development. Any drug entering pre-clinical development can be tested in the humanized BAC mice to more clearly assess whether the drug is likely to induce the CYP450 and MDR1 system in humans. In addition, efficacy studies will be more relevant in this mouse because the drug's metabolism will more accurately reflect its actions in humans.

Since some anti-microbial agents are known to dramatically stimulate the induction of CYP450 system and MDR1 in humans and not in rodents, a humanized PXR BAC mouse could be particularly important for developing of novel antibiotics and determining whether new antibiotics cause significant induction of the CYP450 system.

There is a critical need for the development of new antibiotics because of the growing incidence of drug resistance bacteria. For example, Neuhauser M et al. ((2003) Antibiotic resistance among gram-negative bacilli in US intensive care units: implications for fluoroquinolone use. JAMA. 19:885-8) reported the susceptibility of bacteria to the widely used antibiotic Ciprofloxacin decreased from 86% in 1994 to 76% in 2000 in the US. This is particularly important because Ciprofloxacin is the main drug used to treat the biological warfare agent anthrax.

The BAC-humanized mouse model is useful in development of novel therapeutics to treat biological warfare agents. First, it would provide a system that is a better predictor of drug-drug interactions in human than presently available. Secondly, as new animal models are developed to test the efficacy of drugs to treat biological warfare agents, the BAC-humanized mice could be employed in those models. Importantly, if genetic models are developed to test new drugs to treat biological warfare infections, the BAC-humanized mice could incorporate those genetic modifications so that efficacy, toxicity and metabolism of the new drug could be tested in the same animal.

A “humanized” animal, as used herein refers to a mouse, or other nonhuman animal, that has a composite genetic structure that retains gene sequences of the mouse or other nonhuman animal, in addition to one or more gene and or gene regulatory sequences of the original genetic makeup having been replaced with analogous human sequences.

“BAC,” as used herein, stands for bacterial artificial chromosome. The invention provides a BAC cloning system. The vector, pBAC, based on the Escherichia coli single-copy plasmid F-factor can maintain complex genomic DNA as large as 350 kb in the form of BACs (see Shizuya and Hosein-Mehr, 2001 for review). Analysis and characterization of thousands of BACs indicate that BACs are much more stable than cosmids or yeast artificial chromosomes (YACs). Further, evidence suggests that BAC clones represent the human genome far more accurately than cosmids or YACs. Because of this capacity and stability of genomic DNA in E. coli, BACs are now widely used by many scientists in sequencing efforts as well as in studies in genomics and functional genomics.

In an illustrative example, the invention provides a method of generating a humanized animal, the method involving recombining a first DNA construct with a non-human animal DNA sequence contained therein with a second DNA construct. The second DNA construct has a human DNA sequence that is flanked by a first and a second non-human animal DNA sequence. In another embodiment, the human DNA sequence is flanked by human sequences. In still another embodiment, the second construct is a DNA construct containing a non-human animal DNA sequence flanked by human sequences is generated. In one embodiment, the sequences are derived from the same non-human animal as is desired to be constructed with the methods of the invention. Exemplary BACs of the invention include, but are not limited to: pBAC108L (ATCC Accession No. U511140) and pBeloBAC11 (ATCC Accession No. U51113).

The first recombination step is carried out in a strain of E. coli that is deficient for sbcB, sbcC, recB, recC or recD activity and has a temperature sensitive mutation in recA. After the recombination step, a recombined DNA construct is isolated, the construct having a human DNA sequence flanked by the first and second non-human animal DNA sequences; a human DNA sequence flanked by human sequences; or a non-human animal DNA sequence flanked by human sequences. The recombined construct is then introduced into a non-human embryogenic stem cell.

The recombined construct can be linearized prior to recombination. In one embodiment, the constructs are linearized prior to introduction into the E. coli cells. When the second construct contains a selection marker, E. coli cells containing unrecombined vectors can be eliminated.

The second DNA construct also can carry positive and/or negative selection markers that can interrupt the human DNA sequence.

The regions flanking the coding DNA sequences utilized in the invention should be a length that allows for homologous recombination. For example, in E. Coli, the minimal flanking region length is about 1-2 kb for a high frequency of recombination. Smaller flanking region length can be used, however it may result in a lower frequency of recombination. For example, the flanking regions may be from about 0.1 to 200 kb, and typically from about 1 or 2 kb to 20 kb.

Embryogenic stem (ES) cells from the non-human animal can be selected for recombinants by including positive and/or negative selection markers in the recombined DNA vector. The ES cells are then introduced into a blastocyst of a nonhuman animal. The chimeric blastocyst then can be introduced into a pseudopregnant host animal to generate a humanized non-human animal. Other methods for generating embryos from ES cells also can be used with the methods of the invention.

The various DNA constructs are selected as appropriate for the size of DNA inserted in the construct. In one embodiment, the first and second DNA constructs are bacterial artificial chromosomes or fragments thereof. In another embodiment, the first and second DNA constructs are linearized prior to recombination in the E. coli cell.

In still another embodiment, the human DNA sequence is a human gene sequence encoding a human gene, having at least one intron contained therein. The vectors can be engineered such that the one intron can have a selection marker encoded within the intron. When a selection marker is included, clones undergoing a desired recombination event can be selected using an appropriate antibiotic or drug.

Human gene sequences utilized in the invention may include, but are not limited to, genes encoding G-protein coupled receptors, kinases, phosphatases, ion channels, nuclear receptors, oncogenes, cancer suppressor genes, viral and bacterial receptors, P450 genes, insulin receptors immunoglobins metabolic pathway genes, transcription factors, hormone receptors, cytokines, cell signaling pathway genes and cell cycle genes.

“G-protein coupled receptors,” as used herein are receptors, the binding of which mediates the cellular responses to a diverse group of signaling molecules, including, but not limited to hormones, neurotransmitters, and local mediators. Such signaling molecules may be proteins and small peptides, as well as amino acid and fatty acid derivatives. All known G protein-coupled receptors have a similar structure of a single polypeptide chain that threads back and forth across the lipid bilayer seven times. G protein-coupled receptors utilize the G proteins by means of which they broadcast into the interior of the cell the message that an extracellular ligand is present.

Kinases are enzymes that catalyze the transfer of phosphate groups from a high-energy phosphate-containing molecule (as ATP or ADP) to a substrate. Kinases utilized in the invention may include, but are not limited to: EGFR, P13K, MAP-kinase, and Akt.

Phosphatases are enzymes that accelerate the hydrolysis and synthesis of organic esters of phosphoric acid and the transfer of phosphate groups to other compounds. Phosphatases utilized in the invention may include, but are not limited to: PTPα, SHP1, SHP2 and CD45.

Ion channels are pores in a cell membrane that allows the passage of specific charged molecules by means of which electrical current passes in and out of the cell. The passage of the ions is allowed in response to a stimulus. Ion channels are proteins. Ion channels are classified by the ions they allow to pass and the stimulus. Examples of ions allowed through ion channels include, but are not limited to potassium ions, sodium ions and calcium ions.

Nuclear receptors are proteins that are present in the nucleus and can bind to hormones. As such, nuclear receptors are important as regulators located in the nucleus of a cell involved in a variety of physiological functions and therefore connected with diseases such as cancer, diabetes or hormone resistance. Nuclear receptors utilized in the invention may include, but are not limited to: TRR, ANDR and GCR.

“Oncogene,” as used herein, refers to a gene or genes that normally play a role in the growth of cells but, when overexpressed or mutated, can foster the growth of cancer. Examples can include, but are not limited to: N-myc, c-myc, erb-B, Her2, neu, ras, ABL, RASK, int, fig, Lck, and fos.

Cancer suppressor genes are genes that normally restrain cell growth but, when missing or inactivated by mutation, allow cells to grow uncontrolled. Accordingly, mutations in tumor suppressor genes that are associated with tumorigenesis generally cause loss of function and release this restraint.

Viral and bacterial receptors are the entry points on a cell where the virus or bacteria can enter the target cell. Such receptors utilized in the invention may include, but are not limited to: Human hepatitis B and C, HIV, M. tuberculosis.

P450 genes encode the proteins responsible for the metabolism of drugs in the body, as discussed above. These enzymes inactivate hormones, small molecule drugs, toxins, and environmental chemicals by making them more polar so they can be eliminated. They are also the major sites for drug-drug interactions. Exemplary P450 genes may include, but are not limited to: CYP3A4, CYP2B6 and CYP2C9.

Insulin receptors are receptors that extend through the cell membrane of a target cell that allow the cell to join or bind with insulin that is in the blood. When the cell and insulin bind together, the cell can take glucose (sugar) from the blood and use it for energy.

Immunoglobins are proteins produced by plasma cells, which are designed to control the immune response in extracellular fluids by binding to substances in the body that are recognized as foreign antigens. Immunoglobulins are grouped by structure and activity. The five classes of immunoglobulins are IgA, IgD, IgE, IgG and IgM. Each Ig unit is made up of two heavy chains and two light chains and has two antigen-binding sites.

As used herein, “metabolic pathway genes” are genes involved a metabolic pathway, which is a series of chemical reactions catalyzed by enzymes in a living system. Generally the pathway either breaks down a large compound into smaller units (catabolism) or synthesizes more complex molecules from smaller ones (anabolism). The product of one reaction in a pathway serves as the substrate for the following reaction. The final products of the pathways have vital functions in the living system. Examples of metabolic pathways include, but are not limited to glycolysis and the Kreb's cycle. In addition, polyketide synthases are an example of a gene cluster.

“Transcription factors” as used herein refer to proteins that recognize and bind to specific DNA sequences associated with a particular gene, and can switch the gene on or off. Gene expression is therefore controlled by the availability and activity of different transcription factors. A number of diseases and disorders are known to result from the disruption of gene expression caused by the absence or malfunction of transcription factors. Transcription factors help synthesize RNA using a DNA template. Exemplary transcription factors may include, but are not limited to: NF-κB, AP-1, Sp-1, Oct-1 and TFIID.

“Hormone receptors” are receptors on a cells' surface that recognize and bind with specific hormones. Various forms of nuclear hormone receptors mediate various processes in the body, such that hormone receptors can be involved with diseases such as diabetes and cancer. PXR, as set forth above, is a hormone receptor which begins the body's response to unfamiliar chemicals and is therefore involved in drug-drug interactions and drug metabolism.

As used herein, “cytokines” are relatively low molecular mass proteins secreted by many different cell types, usually consisting of a single chain. Cytokines are signaling molecules that activate other cells, coordinate, and regulate biological processes such as cell growth and immunity. In many ways, cytokines are similar to hormones. Exemplary cytokines include, but are not limited to interferon-a, interferon-b, tumor necrosis factor (TNF), granulocyte colony stimulating factor (G-CSF), platelet-activating factor (PAF), lymphokines, interleukins (IL) and monokines.

Cell signaling pathways, as used herein are the means by which individual cells of an organism communicate, in order to coordinate their behavior. Cell signaling is at the core of most biological processes. Cell-signaling systems may include, but are not limited to cell-surface and intracellular receptor proteins, protein kinases, protein phosphatases and GTP-binding proteins. “Cell signaling pathway genes” are genes involved in such pathways.

The “cell cycle,” as used herein, refers to the events that result in cell growth and division of a cell into two daughter cells. The cell cycle involves the S phase, the G2 phase, the M phase and the G1 phase. Cell cycle genes are genes involved in or that regulate the cell cycle. Cell cycle genes can include, but are not limited to Cdk, MPF and p53.

One or more additional selection markers can be added following the recombining step to the recombined construct. In one embodiment, a positive selection marker is added within an intron in the human DNA sequence. In yet another embodiment, a negative selection marker is added to a position flanking either of the non-human DNA sequences.

The methods of the invention can be used with any non-human animal for which ES cells are available. In one embodiment, the ES cells are mouse ES cells and the non-human animal is a mouse, and the methods of the invention are used to create a humanized mouse.

The methods of the invention can be used to precisely determine the joints between the human and non-human sequences. In one embodiment, only the coding sequence of the non-human animal is humanized. In such an embodiment, the first non-human DNA sequence in the second construct is joined at the 5′ of a start codon of the human gene coding sequence and the second non-human DNA sequence in the second construct is joined to the 3′ of a stop codon of the human gene coding sequence. In another embodiment, only the regulatory (control) sequence of the non-human animal is humanized. In still another embodiment, both the coding and the regulatory (control) sequences of the non-human animal are humanized.

The human DNA sequence to be used can be a human genomic sequence or can be a non-natural sequence encoding a human gene product. In one embodiment, the sequence is a non-natural sequence that encodes a human gene product, but has been codon-optimized for improved expression in the non-human animal. In another embodiment, the sequence is a chimeric gene that incorporates certain human exons but retains some non-human exons. In still another embodiment, the sequence is a chimeric gene that has some or all human exons, but keeps some or all non-human introns.

The invention also provides a DNA construct for performing homologous recombination within a cell, having a human DNA coding sequence with at least one intron and a selection marker gene contained within the at least one intron. The construct also has first and second non-human animal DNA sequences flanking the human DNA. The non-human animal flanking sequences are homologous to sequences in the genome of the non-human animal that flank a gene orthologous to the human DNA coding sequence. In one embodiment, recombination in an ES cell directs replacement of the non-human gene with its human orthologue. Additionally, or alternatively, the construct may have human flanking sequences or may have a non-human animal DNA sequence flanked by human sequences.

In another embodiment, the DNA construct also has a second selection marker adjacent to one of the non-human DNA sequences. In an embodiment, the construct is a bacterial artificial chromosome. In another embodiment, the construct is linearized. In one embodiment, when the DNA construct is to replace a mouse gene, the first and second non-human DNA sequences are mouse genomic DNA sequences. In another embodiment, the non-human sequences can be joined adjacent to the human gene coding region, or can be joined outside the coding region. In another embodiment, the non-human sequences are joined to the human sequence outside the coding region and including some or all of the 5′ and 3′ regulatory or control DNA sequences, including for example, promoter and enhancer sequences. Therefore, the non-human sequences can be joined to the human sequence adjacent to the 5′ end of the start codon or adjacent to the 3′ end of the stop codon.

In one embodiment of the invention, a first DNA vector is constructed that has human DNA flanked by mouse DNA. The DNA vector can be any suitable DNA vector, including a plasmid, BAC, YAC or PAC. In one embodiment, the DNA vector is a bacterial artificial chromosome.

As used herein, the term “vector” refers to a nucleic acid molecule into which another nucleic acid fragment can be integrated without loss of the vector's ability to self-replicate. Vectors may originate from a virus, a plasmid or the cell of a higher organism. Vectors are utilized to introduce foreign DNA into a host cell, wherein the vector is replicated.

The term “construct,” as used herein refers to a sequence of DNA artificially constructed by genetic engineering or recombineering.

A polynucleotide agent can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, alpha virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb 25:37-42, 1993; Kirshenbaum et al., J. Clin. Invest 92:381-387, 1993; each of which is incorporated herein by reference).

A DNA vector utilized in the methods of the invention can contain positive and negative selection markers. Positive and negative markers can be genes that when expressed confer antibiotic resistance to cells expressing these genes. Suitable selection markers can include, but are not limited to: Km (Kanamycin resistant gene), tetA (tetracycline resistant gene) and G418 (neomycin resistant gene). The selection markers also can be metabolic genes that can convert a substance into a toxic substance. For example, the gene thymidine kinase when expressed converts the drug gancyclovir into a toxic product. Thus, treatment of cells with gancylcovir can negatively select for genes that do not express thymidine kinase.

In one embodiment of the invention, the first DNA vector is generated by PCR using two BAC vectors, one containing DNA for a human gene and the second for a mouse gene. As used herein, “gene” can refer to a wild-type allele (including naturally occurring polymorphisms) and mutant or engineered alleles. In one embodiment, an allele is engineered to encode a naturally-occurring human allele, but the DNA sequence has been codon optimized to reelect the codon preferences of the non-human organism. Codon preferences are well known to one of skill in the art. The genes utilized in the invention may be, for example, gene coding sequences or gene regulatory regions.

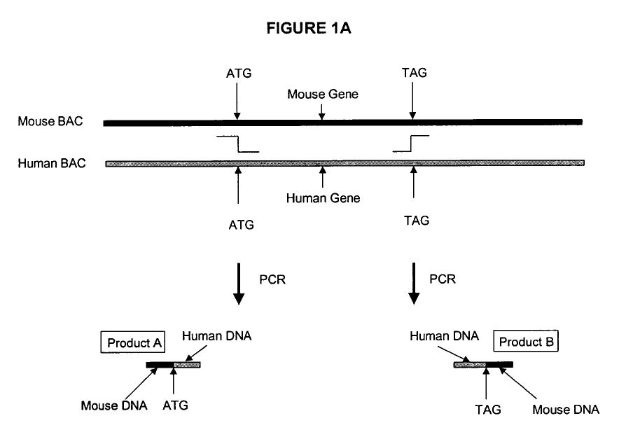

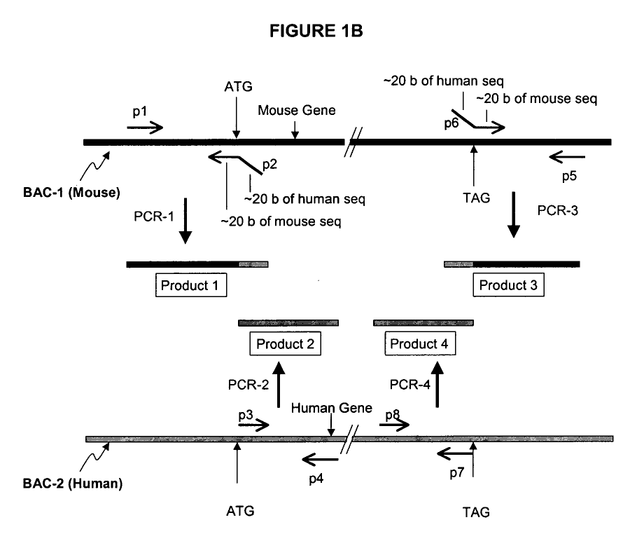

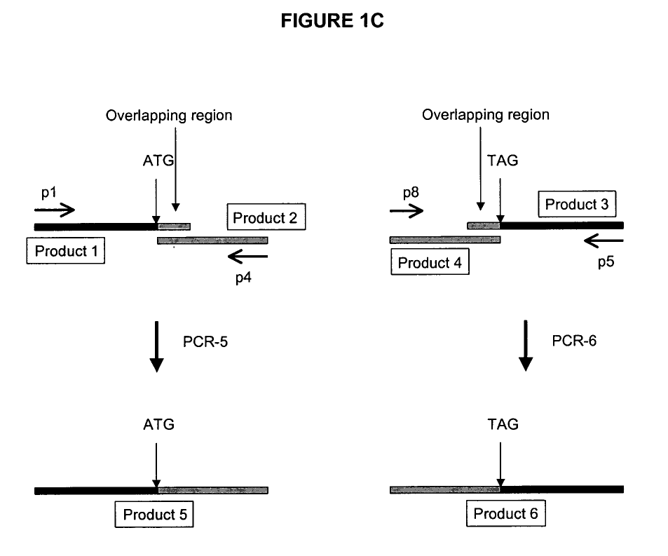

FIG. 1A shows the PCR procedure used to generate recombinant DNA between mouse and human sequences. Two BACs carrying either mouse (BAC-1) or the human orthologue of the mouse gene (BAC-2) gene are created. The BACs may include the control region contiguous to the coding region. Two PCR products (pA and pB) are made; both are hybrid products between human and mouse DNA. The first half of pA is 2 kb upstream of mouse DNA from the beginning of the coding region and the second half is 2 kb human DNA starting at the first codon ATG of the human coding region. Likewise, the half of pB is 2 kb human DNA containing the last codon TAG at the junction of the second half that is 2 kb downstream of mouse DNA from the TAG. More detailed description of the PCR is shown in FIGS. 1B and 1C.

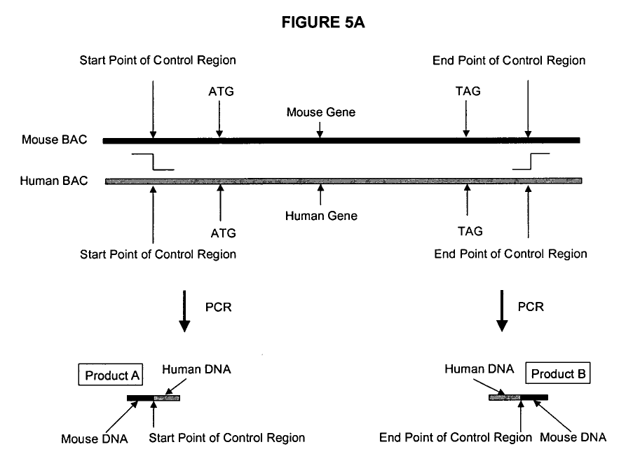

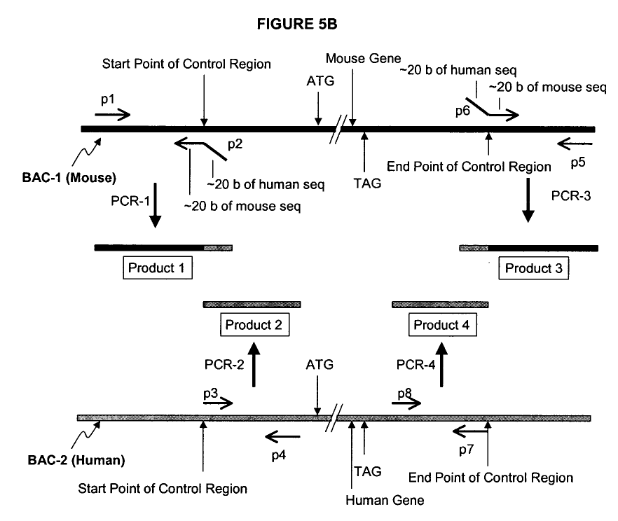

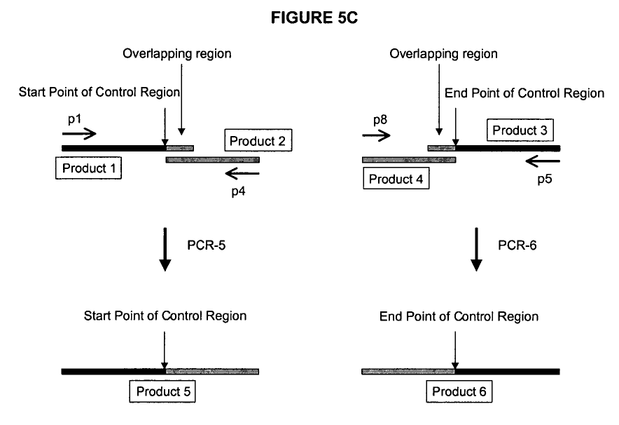

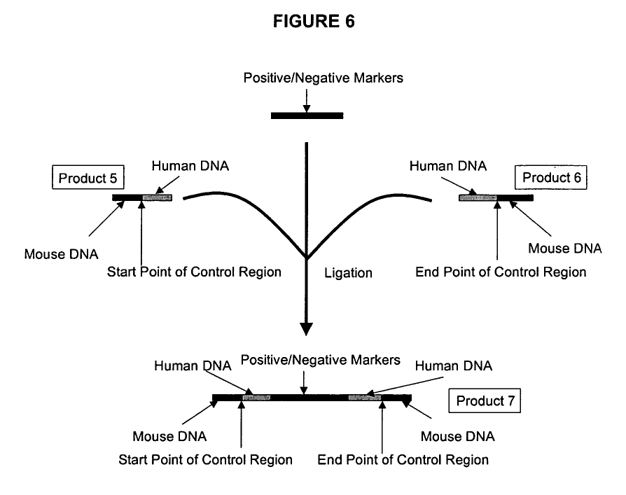

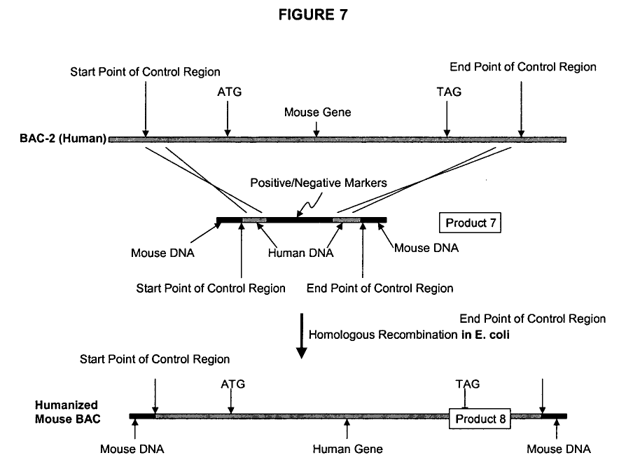

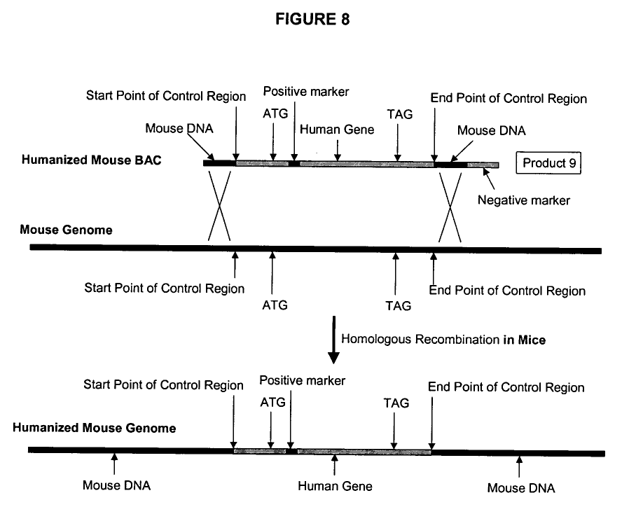

FIG. 1B, shows PCR-1 carried out using primer-p1, which is ˜20 bases long derived from the end of 2 kb region that is upstream from the first amino acid codon ATG and the other primer-p2 that has ˜40 base hybrid sequence: the first half (5′ end) sequence of p2 contains first 20 bases of human coding region ending at ATG and the second half contains ˜20 base mouse DNA upstream from the ATG codon. The PCR product (Product 1) is thus a hybrid between human and mouse DNA, containing ˜20 base human DNA and about 2 kbp of mouse DNA. Product 3 contains the last 20 bases including the stop codon TAG of human coding region and about 2 kb of downstream region of mouse BAC DNA. Products 2 and 4 are ˜2 kb in length, each of which contains ATG and TAG of human coding regions respectively. As shown in FIG. 1B, primers can be used that generate DNA fragments that correspond to the junction of coding and non-coding regions of the gene. It is also possible to choose the junctions to include regulatory sequence regions to either or both of the 3′ and 5′ ends of the gene. For example, FIGS. 5-8 illustrate an example in which the desired regions flank the coding region of a gene to include regulator sequences both to the 5′ and 3′ of the gene. In FIG. 5, two BACs carrying either mouse (Mouse BAC) or human (Human BAC) gene that is an orthologue are used. The BACs include the control region contiguous to the coding region. Two PCR products (Product A and Product B) are made; both are hybrid products between human and mouse DNA. The first half of pA is about 2 kb upstream of mouse DNA from the beginning of the control region and the second half is about 2 kb human DNA starting at the beginning of the control region of the human coding region. Likewise, the half of product B is 2 kb human DNA containing the end of a desired region of the 3′ control region and the second half that is 2 kb downstream of mouse DNA from the end of the orthologous mouse control region.

A second round of PCR can be used to generate PCR products having DNA from both mouse and human. FIG. 1C, for example, shows the use of PCR primers to generate fragments labeled Product 5 and Product 6 that have a junction between the human and mouse DNA at the ends of the coding region of the gene. As shown in FIG. 1C, there is an overlapping 20 bases between 3′ end of Product 1 and 5′ end of Product 2. Using primers p1 and p4, and the two product, PCR-5 generate ˜4 kb Product 5 that is a fused DNA at the overlapping region. Likewise, ˜4 kb Product 6 is generated as a fused DNA between Products 3 and 4.

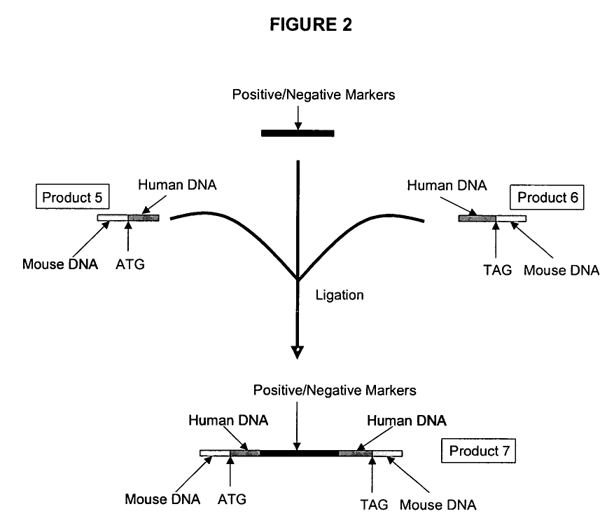

FIG. 2 illustrates an assembly of the Products 5, 6 and positive/negative markers by a three part ligation reaction. Only those constructs that include the positive selection marker will grow in the presence of an antibiotic present in the medium in which bacteria transformed with the construct are grown. The resulting construct, illustrated as Product 7 has positive and negative markers flanked by human DNA sequences and further flanked by mouse DNA sequences.

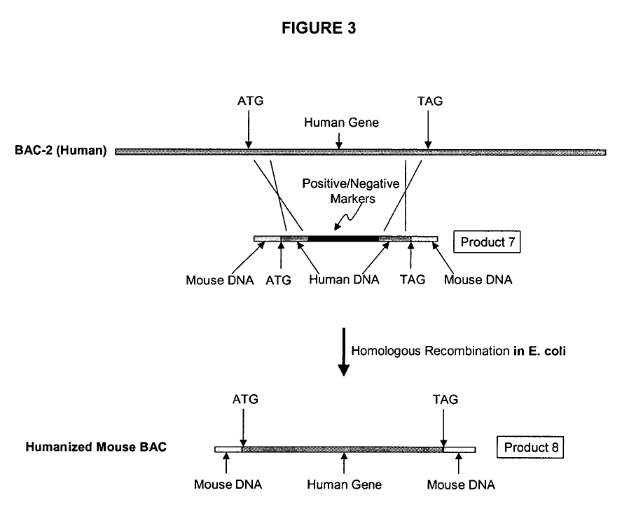

This construct, Product 7, can be linearized and introduced into E. coli cells that are deficient for recB, recC or recD as well as deficient for sbcB and sbcC and are temperature sensitive in recA. General recombination between a BAC having the corresponding human gene sequence (BAC-2) and linearized Product 7 is carried out in E. coli strain (FIG. 3). Because of recA ts, the electro-competent cells are prepared by growing at 300 C (permissive temperature for recA ts in general recombination). After electroporation into the strain having both already existing BAC-2 and incoming Product 7, transformed cells are incubated at 42° C. (non-permissive temperature for recA ts) under an appropriate condition for selecting the desired recombinant. The resulting Product 8 is modified BAC-1 whose mouse gene is replaced by the corresponding human gene.

In one embodiment, Product 8 is modified with a positive marker gene that is situated within an intron of the human gene as well as with a negative marker flanking at least one side of the Product 8 to give new Product 9. In one embodiment, the positive selection marker used is G418 (neomycin resistant gene) and the negative marker is TK (thymidine kinase gene).

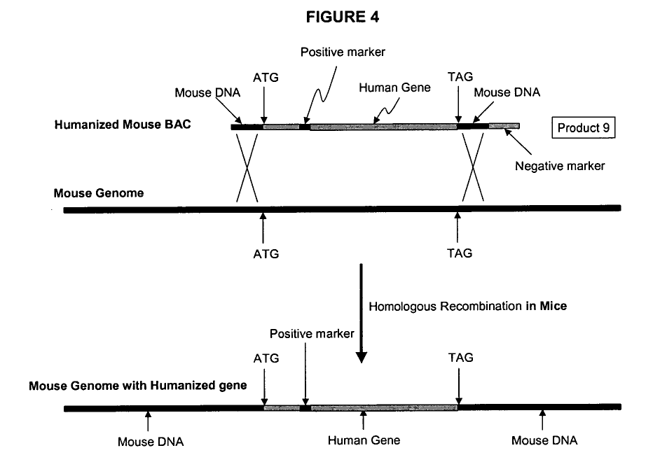

Mouse embryogenic stem (ES) cells are transformed with the humanized mouse BAC, Product 9 (FIG. 4). ES cells are selected that have Product 9, which are those having the positive selection marker (are neomycin resistant) and lacking the negative selection marker (are insensitive to gancyclovir). The resultant recombinants are used to implant mice.

ES cells can be implanted into mouse blastocysts which can then be transferred to pseudopregnant female mice who can carry the mice to term. In one embodiment, the ES cells are of a distinct genetic background from the surrogate mice. Such differences, for example in coat color, allow for the rapid identification of mice having incorporated the ES cell.

Two cycles of general recombination are performed. In the first cycle, general recombination is carried out in a strain of Escherichia coli, which is disabled in the recB, recC, sbcB and sbcC, for example, through a knock-out mutation, and has a temperature sensitive mutation in recA.

In the second cycle, general recombination is taken place in either ES cells or eggs. Humanized BAC replace the corresponding mouse gene in the mouse genomic by general recombination.

Almost all proteins found in mammalian cells interact with themselves and/or other proteins. Proteins function together in a pathway and the proteins take part in activities to perform related biochemical tasks. Mice and humans have homologous genes for a given pathway. However, when one mouse gene is humanized, then the association between the humanized protein and the mouse proteins may fail to occur correctly. To create a fully functional humanized mouse in the pathway, all or most of the mouse genes involved in the association must be replaced with the corresponding human genes.

For example, where there are two interacting proteins, and one is an enzyme endo-peptidase while the other is its substrate protein, the enzyme recognizes the specific site localized in the substrate protein, and makes an incision at the site to split the protein into two portions. When the substrate protein is humanized, the mouse peptidase may no longer be able to recognize the site and the proper incision may not occur at the human protein site. This can be corrected by humanizing the mouse endopeptidase gene.

Comparative DNA sequence analysis of the human genome has revealed a large number of single nucleotide polymorphisms (SNPs) dispersed over 3 billion bases of the human genome. Some SNPs do not change the amino acid of the gene and others, while changing the amino acid of the gene do not alter the function of the gene. Other SNPs cause significant consequence on the function of the gene, sometimes resulting in severely altered phenotype. Most of the drugs are developed using the wild type or the most common form of human proteins, not the mutated form of the protein. When such a drug is administered to patients having presumably the altered gene, the drug may not work as expected in individuals because of the different form of the target proteins.

These human polymorphisms must be linked to pharmacokinetic profiles for each drug candidate. The profiles can be made by using humanized mice carrying mutated forms of human genes. This type of humanized mouse shares the same genetic background as a non-altered mouse, except for the human originated gene. In drug profile studies, the environmental and non-genetic conditions that can often interfere and affect drug response and metabolism are controlled and set at the identical condition for all humanized and non-humanized mice. The only difference among the animals is the genotype and thus the results of drug evaluation and metabolism can be directly compared and evaluated, generating highly accurate and reliable profiles.

A number of non-infectious human diseases have no validated animal models for clinical evaluation. These diseases are often associated with genetic polymorphism in allelic mutations. humanized mice created by the incorporation of the alleles relevant for a particular disease can be a valuable model for monitoring the disease development and subsequent evaluation of drug efficacy.

In the arena of infectious disease, most of the human viruses do not infect non-primate experimental animals such as mice and rats. One reason for this lack of infection may be an intracellular block of the infection. For example, VSVg pseudo-types EIA virus (an equine lentivirus) readily enters human cells, but cannot undergo a productive replication cycle because certain species-specific cellular factors are absent. Another reason for the lack of infection may be because only the human receptor(s) can interact with human viruses and vice versa. However, once the mice are humanized by replacing the corresponding mouse receptors or factor with the human orthologue, the humanized mouse is expected to present the same progression of human viral disease upon infection. This approach enables the creation of a mouse model for human viral infectious diseases and for evaluating the response of antiviral drug and vaccine for humans in the living humanized mice.

Through the use of BAC engineering, a humanized mouse is created by replacing mouse target genes with the corresponding human genes in their entirety. Because of this replacement, only the human genes in the manipulated region will be functionally expressed in the living humanized mouse. An array of humanized mice will be created expressing various human genes relevant to drug evaluation and toxicity screening. humanized mice will make it possible to obtain more direct assessment on how well and how safe the drugs in development will work in human. The assessment will then lead to rapid decisions for potential drug candidates at an early developmental stage.

The utility of humanized mice can also be extended to establish new animal models for monitoring the progress of human diseases and the subsequent development of therapeutic drugs. Furthermore, various alleles of the human genes can be introduced into humanized mice for assessing drug response of people with genetic polymorphism.

The invention allows for natural tissue specific expression of genes, including splice variants, at physiology levels and under normal regulation that can not be achieved with any other transgenic (cDNA) technologies. This capability is due to the ability of BACs, through homologous recombination, to precisely integrate human sequences of almost unlimited size into the corresponding mouse genome. These transferred human sequences may include many if not all of the 5′ and 3′ regulatory regions of the human genes, or alternatively, be limited to the coding region (including introns) to allow for regulatory control by the endogenous mouse regulatory region.

The examples set forth below provide the basis for generation of a mouse that responds like humans to drug inducers of the CYP450 system and P-glycoprotein. Specifically, in Example 1, BAC is used to express human PXR in mice in the appropriate tissue locations and under normal physiological control. In Example 2, the transformed mice are tested for whether they respond appropriately to drugs known to induce the human CYP450 system but which are inactive in the wild-type mouse. Because of the power of the BAC system, additional human genes can be inserted into the mice already humanized and expressing the human PXR gene.

The humanized mouse PXR system developed in this invention is important for developing new therapeutics to counter the threat of bioterrorism since it is known that the most effective stimulants of the human CYP450 system are the anti-microbials, rifampicin and clotrimazole, which do not affect the mouse CYP450 system. The mice can be used to predict whether new antibiotics being developed to treat biological warfare agents will cause drug-drug interactions in humans. Most importantly, if genetic models are created to facilitate the development of new anti-biowarfare drugs, those models can be incorporated into humanized mice to provide a fully integrated system to develop efficacious and safe Biodefense therapeutics.

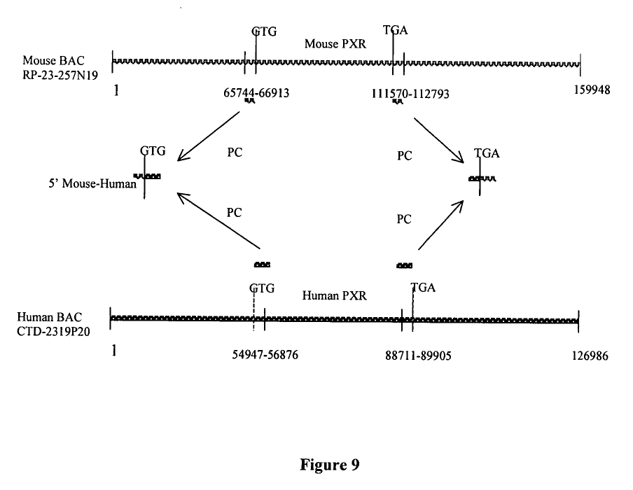

To generate the humanized PXR mouse, two BACs (human and mouse) are required to complete the construction of humanized mouse PXR BAC. Human BAC CTD-2319P20 covers the region starting at 119,074,966 and ending at 119,201,951 of human chromosome 3q13.33. Human PXR genomic coding portion encompass the segment of 54,947 to 89,905 in 2319P20 BAC (length=126,986 bp) (FIG. 9). Mouse BAC (RPC23-257N19) is 159,948 bp long and localized at the region from 38,010,752 to 38,170,699 of mouse chromosome 16. Mouse PXR genomic coding segment is from 66,913 to 111,570 of 257N19 BAC (FIG. 5).

As outlined in FIGS. 9 to 11, the construction of a pair of head and tail chimeras, and the subsequent fusion product has been completed. The head chimera is derived from 1,169 bp upstream region of the first codon GTG of mouse PXR and from 1,929 bp downstream region of the first codon GTG of human PXR. This chimera has been made by a two-step PCR procedure (in all of the PCR experiments, Herculase polymerase is used to significantly reduce the mutation rate during PCR cycles); the first PCR generated 1,169 bp and 1,929 bp products from corresponding regions, and the second PCR has generated the chimera product via 40 bp overlapping segment between the two initial products. The resultant product is called 5′ head chimera. Likewise, 3′ tail chimera has been constructed by the fusion of 1,194 bp human and 1,223 bp mouse segments (FIG. 9). The last terminator codon TGA is at the junction of two segments as illustrated in FIG. 9.

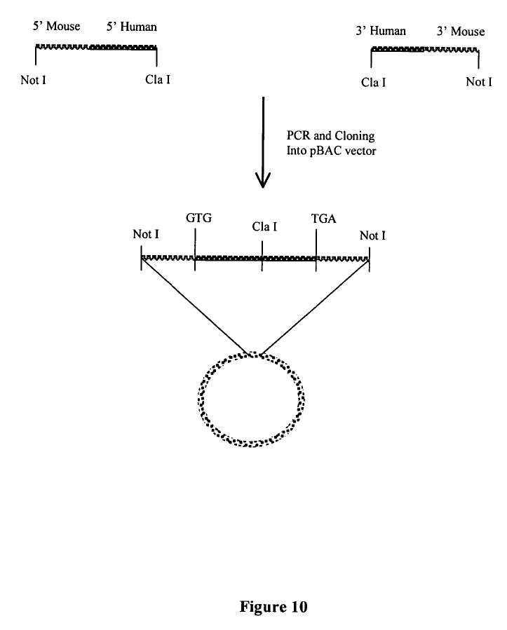

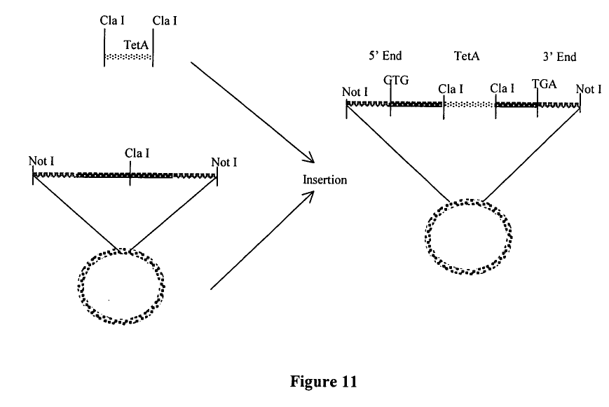

The 5′ head and 3′ tail chimeras were merged by similar fusion PCR using a short overlapping segment between 5′ and 3′ human segments as shown in FIG. 10. The resultant 5.5 kb fragment was cloned into pBAC vector, and then the tetA gene was inserted into the Cla I site (FIG. 11). The final product is 14.2 kb consisting of chimeric head and tail, and the positive selection marker tetA.

Further studies will expand the limits of the humanized mice to reproduce a human drug metabolism system to serve as an animal model to measure of drug-drug interactions. The method of the invention will be used to generate mice co-expressing human PXR and CAR, a major regulator of the expression of CYP2B genes responsible for mediating phenobarbital induction of CYP450 enzymes and like PXR, with significant species variations in amino acid sequences and drug sensitivity. The power of the BAC system will enable generation of even larger human gene networks in the mice by co-expressing human RXR, which serves as a co-factor with PXR in regulating CYP450 genes and in addition, insert the human CYP450 genes themselves, with their unique regulatory regions (while knocking out the mouse counterparts) to generate a fully integrated human P450 system.

Screening of most available anti-microbial agents would then begin to assess their ability to induce CYP450 and MDR1 expression in these humanized mice to determine their potential for drug-drug interaction. This would serve at least two purposes. First, it will further validate the utility of the mouse system for evaluation of drug-drug interaction, since most antibiotics and anti-viral drugs have been tested for P450 induction and drug-drug in humans, and therefore comparison of results in humans with humanized mice prepared by the method of the invention will determine how closely the animal model can predict effects of the anti-microbial agents. And secondly, such studies will serve as the foundation for establishing the use of the humanized mice to screen new antibiotics and anti-viral agents being developed to treat infection and biological warfare agents at an early stage to determine their potential side-effects in humans. This will help to prioritize the development of those therapeutics which are least likely to cause P450 or MDR1 induction and other complications in humans.

The following examples are intended to illustrate but not limit the invention.

### EXAMPLE 1

### Development of a Mouse Expressing Human PXR Using BAC

Using BAC technology the entire mouse PXR coding region will be replaced with the corresponding human PXR coding region (including introns) by homologous recombination. Human PXR gene expression will be detected in the transformed mice by Northern analysis and PCR. Particular attention will be made to determine whether human PXR is expressed in liver and gastrointestinal tract, and other tissues that normally express the receptor in humans. This will distinguish this approach from any other transgenic procedures used to express human PXR in mice.

First, an E. coli host is needed that has certain characteristics that allow stable propagation of large mammalian DNA inserts in the BAC vector, and is able to selectively carry out proper homologous recombination when needed. The strain HS996, which will be used for these studies, has been constructed to accommodate large BAC inserts, and its recA⁺ derivative HS985 has been chosen as a founder strain for further modification. This strain has been modified to perform conditional homologous recombination; cells will become proficient in recombination only when cells are grown at 30° C.

The relevant genotypes of HS985 for the work are: RecB21, recC22, sbcB15, sbcC201, mcrA⁻, del(mrr-mcrBC), and endA1. Mutations in RecB, C and endA1 allow E. coli to protect incoming linear DNA from degradation. Mutations in sbcB and C inhibit degradation of DNA having hairpin structure. Mutation in mcrA⁻ and del(mrr-mcrBC) remove the host restriction-modification system, therefore mammalian DNA is not degraded.

RecAts200 is a temperature sensitive mutant for generalized recombination. Mutation of recAts200 has been introduced to HS985 by P1 transduction. Phage P1 grown in a strain carrying recAts200 has prepared and infected into HS985 to obtain recombinant clones having the phenotype of temperature sensitive recombination. The resultant strain HS2001 has been further tested to confirm the genotype of HS985.

HS2001 is defective in recombination at high temperatures (40° C.) whereas at lower temperature (30° C.) it is capable of carrying out recombination normally. For the BAC DNA transfection studies, electrocompetent HS2001 prepared at 30° C. is used and the transfected cells are allowed to grow at 30° C. until the recombination is finished, and then raise the temperature to 40° C. to prevent unwanted recombination events, which can include the formation of deletions and rearrangement due to repeated DNA sequences often found in mammalian DNA. It has been shown that the deletion and rearrangement of BAC DNA are extremely rare in recA mutants (Shizuya H. et al. (1992) Cloning and stable maintenance of 300 kb-pair fragments of human DNA in E. coli using F-factory based vector. PNAS 89:8794-8797.), and thus at 40° C. virtually no unwanted recombination in HS2001 strain is expected.

As outlined in FIGS. 9 to 11 the BAC-human PXR construct DNA has already been generated. The next step will be the transfection of the BAC-PXR construct DNA into ES cells. For this, approximately 10 million C57BL/6 ES cells will be transfected with BAC-PXR construct DNA. Transfected ES cells will then be cultured on embryonic fibroblast feeder layers in presence of G418 for a period of up to 2 weeks. Up to five hundred G418 resistant C57BL/6 ES clones will be isolated and expanded for individual genomic DNA isolation and generation of frozen cell stocks. Primary Southern blot analysis will be performed to select targeted clones and up to four selected primary clones will be expanded for large-scale DNA preps and additional frozen stocks. Secondary Southern blot analysis will be performed on the primary targeted clones with multiple enzymes and multiple probes (5′, 3′ and neo probe) to confirm homologous recombination events at the target locus. Karyotypic analysis of up to three secondary clones will be used to identify the most suitable clone(s) for expansion for microinjection.

Once the ES cells are generated, chimeric mice will be generated. In order to facilitate screening of chimeric mice, the C57BL/6 “black” mouse ES cells generated will be injected into FVB “white” mice. Live births from the implanted blastocysts that have incorporated the “black” ES cell will be chimeric for coat color and easily identified. A total of 100 “chimeric” blastocysts will be injected for each clone. Injected blastocysts will be transferred into pseudo-pregnant FVB females for generation of chimeras.

Up to five high percentage coat color chimeras will be bred to C57BL/6 mice in order to maximize the possibility of germ line transmission of the PXR recombinant and transfer of the recombinant genotype to the “black” mouse background. PCR and Southern genotype analysis will be performed on the progeny to identify heterozygotes (F1s). These mice will be cross-bred to obtain homozygous C57BL/6 human PXR (huPXR) mice. Homozygous mice will be identified by PCR and Southern blot analysis and expanded to a colony. The line will be secured by freezing of embryos of cross-bred homozygous C57BL/6 huPXR mice. At this point humanized PXR mice will have been generated.

To test for expression of mouse PXR in null mice, extract RNA will be extracted from liver and small intestine and use Northern blotting to detect mouse PXR mRNA using mouse PXR cDNA probes as described by Xie et al. (2000). In mice expressing the human PXR, RNA will be isolated from liver and small intestine and ³²P-labeled probes will be used against the 1.0 kb fragment encoding the ligand binding domain of human PXR (which differs considerably from mouse PXR) to detect human PXR mRNA as described in Lehmann et al. ((1998) The Human Orphan nuclear receptor PXR is activated by compounds that regulate CYP3A4 gene expression and cause drug interactions. JCI 102:1016-1023.) PCR will be used to verify results from the Northern analysis.

### EXAMPLE 2

### Test Response of Mice Expressing Human PXR to Drugs That Induce CYP450 Expression in Humans

Animals developed in Example 1 will be tested for ability to respond to drugs that induce human CYP450 expression. The drugs to be tested are the anti-microbial drugs rifampicin and clotrimazole. Their abilities to increase the expression of the major CYP450 enzymes will be measured, including CYP3A, CYP2B6 and CYP2C9 in liver and other tissues that normally express PXR in humans by Northern analysis, RNAse protection assays and by ELISA. As a control, the effects of pregnenolone 16α-carbonitrile, a molecule that stimulates mouse PXR to induce CYP450 but does not interact with human PXR will also be tested. Additionally, it will be tested whether these drugs increase the expression of P-glycoprotein (MDR1) a major drug efflux transporter involved in drug elimination under the regulation of human PXR. If the mice respond to drugs that normally stimulate human PXR, the first step in generating a humanized mouse with a fully operational human drug metabolism system that can be predictive of drug-drug interactions in the human will have been accomplished.

For these studies, mice generated in Example 1 will be studied for pharmacological analysis. Mice will be administered rifampicin (5 mg/kg by gavage) for various times (12 hr, 1, 2 and 3 days) and for 3 days at different concentrations (1, 3, 5 and 10 mg/kg by gavage) as described by Xie et al. (2000) to determine its time course and dose-dependency to induce CYP450 gene expression in liver and intestine. Other humanized PXR mice will be treated ip (intraperitoneally) with a single dose of either clotrimazole (50 mg/kg), dexamethasone (50 mg/kg) or pregnenolone-16a-carbonitrile (PCN)(40 mg/kg) for one day. Clotrimazole, like rifampicin selectively stimulates human PXR while dexamethasone and PCN primarily stimulate mouse PXR and will serve as a control for these studies on humanized PXR animals. The effects of these drug treatments on liver and intestine CYP3A mRNA as well as liver mRNAs for CYP2B6, CYP2C9, CYP7A and CYP1A2 will be detected by Northern blot and RNAse protection assays with a β-actin cDNA probes (CLONTECH Laboratories Inc., Palo Alto, Calif.) as a control.

For these studies, after the drug treatments, the mice will be anesthetized with isofluorane and exsanguinated at the time of sacrifice. Immediately following exsanguination, the livers will be perfused via the portal vein using approximately 50 mL ice-cold 1.15% potassium chloride. The liver and small intestine will be dissected and trimmed of fat and other contiguous tissue in a uniform manner. The liver and intestine will be rinsed in ice-cold 1.15% potassium chloride, blotted, and weighed. Immediately after weighing, the liver and intestine will be placed in aluminum foil, appropriately labeled, and transferred to a liquid nitrogen environment for freezing. After freezing in a liquid nitrogen environment, the samples will be placed in an airtight plastic container and maintained on dry ice until stored at approximately −70° C. The frozen livers and intestine will be shipped to Aliva, thawed, homogenized at 4° C., and total RNA will be prepared using TRIZOL Reagent (Gibco, BRL) and Northern analysis will be carried out as described by Xie et al. (2000). Probes for the different CYP450 mRNAs will be cloned by PCR followed by reverse transcription from wild-type mouse liver mRNA. CYP450 protein levels will be measured using commercially available ELISA kits. In these studies, the protein concentration in the tissue under study will be determined with the Biorad Bradford assay. In liver and intestine of the humanized PXR mice, the effect of the drug treatments on MDR1 expression will be measured by Northern blotting using cDNA probes as described in Synold et al. (2001). P-glycoprotein levels will be measured by Western blotting using antisera from Oncogene Research Products (Boston, Mass).

Statistical analyses of critical data that yields pertinent information as to whether the test material caused liver or intestine CYP450 or MDR1 induction will include the following: body weight, protein concentration of liver or intestine preparation, amount of CYP present per gram of tissue protein (when ELISA is used to measure CYP450 levels). Statistical analysis will be made between treatment groups using parametric (e.g., one-way analysis of variance, Dunnett's t test, Student's t test) or non-parametric (e.g., Kruskal-Wallis statistic, Dunn's test, Mann-Whitney U test) statistical procedures. The choice of parametric or non-parametric test will be based on whether the groups to be compared satisfy the homogeneity of variance criterion (evaluated by Bartlett's test or Ftest). Statistical significance will be assumed when p<0.05.

Although the invention has been described with reference to the above examples, it will be understood that modifications and variations are encompassed within the spirit and scope of the invention. Accordingly, the invention is limited only by the following claims.

What is claimed is:

1. A method of generating a humanized animal, comprising:
recombining a first DNA construct with a second DNA construct,
wherein the first DNA construct has a non-human animal DNA sequence contained therein, and wherein the second DNA construct has a human DNA sequence contained therein and the human DNA sequence is flanked by a first and a second non-human animal DNA sequence;
isolating a recombined third DNA construct having a human DNA sequence flanked by the first and second non-human animal DNA sequence; and
introducing the recombined third DNA construct into a non-human embryogenic stem cell.

2. The method of claim 1, further comprising introducing the embryogenic stem cells into a non-human blastocyst and introducing the chimeric blastocyst into a pseudopregnant non-human animal.

3. The method of claim 1, wherein the first DNA construct is a bacterial artificial chromosome.

4. The method of claim 1, wherein the second DNA construct is a bacterial artificial chromosome.

5. The method of claim 4, wherein the bacterial artificial chromosome is linearized.

6. The method of claim 1, wherein the recombining is carried out in a strain of E. coli.

7. The method of claim 1, wherein the E. coli is deficient for sbcB, sbcC, recB, recC or recD activity and has a temperature sensitive mutation in recA.

8. The method of claim 1, wherein the human gene sequence is selected from the group consisting of genes encoding G-protein coupled receptors, kinases, phosphatases, ion channels, nuclear receptors, oncogenes, cancer suppressor genes, viral receptors, bacterial receptors, P450 genes, insulin receptors immunoglobins metabolic pathway genes, transcription factors, hormone receptors, cytokines, cell signaling pathway genes and cell cycle genes.

9. The method of claim 1, wherein the third DNA construct is a bacterial artificial chromosome.

10. The method of claim 1, wherein the human DNA sequence is a human gene sequence having at least one intron contained therein.

11. The method of claim 1, wherein the third DNA construct has a selection marker contained within the intron.

12. The method of claim 11, wherein the selection marker is added following the recombining step.

13. The method of claim 11 wherein the selection marker is a positive selection marker.

14. The method of claim 11, wherein the third DNA construct has a second selection marker that flanks the non-human animal DNA sequence.

15. The method of claim 1, wherein the non-human animal is a mouse and the non-human embryonic stem cells are mouse embryonic stem cells.

16. The method of claim 1, wherein the human DNA sequence and the first non-human DNA sequence in the second construct are joined to the 5′ of a start codon in a human gene coding sequence.

17. The method of claim 16, wherein the human DNA sequence and the second non-human DNA sequence in the second construct are joined to the 3′ of a stop codon in the human gene coding sequence.

18. A DNA construct for performing homologous recombination within a cell, the construct comprising:
a human DNA coding sequence having at least one intron disposed therein;
a selection marker gene contained within said at least one intron;
a first and second non-human animal DNA sequences flanking the human DNA, wherein the non-human animal flanking sequences are homologous to sequences in the genome of the non-human animal that flank a gene orthologous to the human DNA coding sequence.

19. The DNA construct of claim 18, further comprising a second selection marker adjacent to one of the non-human DNA sequences.

20. The DNA construct of claim 18, wherein the construct is a linearized bacterial artificial chromosome.

21. The DNA construct of claim 18, wherein the first and second non-human DNA sequences are mouse genomic DNA sequences.

22. The DNA construct of claim 18, wherein the flanking sequences are from about 0.1 to 200 kb in length.

23. The DNA construct of claim 18, wherein human DNA coding sequences and the first non-human sequence are joined adjacent to the 5′ end of the start codon of the human DNA coding sequence.

24. The DNA construct of claim 18, wherein human DNA coding sequences and the first non-human sequence are joined adjacent to the 3′ end of the stop codon of the human DNA coding sequence.

25. A method for generating a DNA construct for performing homologous recombination within a cell by
recombining a first DNA construct with a second DNA construct,
wherein the first DNA construct has a non-human animal DNA sequence contained therein,
wherein the second DNA construct has a human DNA sequence contained therein and the human DNA sequence is flanked by a first and a second non-human animal DNA sequence;
isolating a recombined third DNA construct having a human DNA sequence flanked by the first and second non-human animal DNA sequence; and
introducing the recombined third DNA construct into a non-human embryogenic stem cell.

26. The method of claim 25, further comprising introducing the embryogenic stem cells into a non-human blastocyst and introducing the chimeric blastocyst into a pseudopregnant non-human animal.

27. The method of claim 25, wherein the first DNA construct is a bacterial artificial chromosome.

28. The method of claim 25, wherein the second DNA construct is a bacterial artificial chromosome.

29. The method of claim 28, wherein the bacterial artificial chromosome is linearized.

30. The method of claim 25, wherein the recombining is carried out in a strain of E. coli.

31. The method of claim 25, wherein the E. coli is deficient for sbcB, sbcC, recB, recC or recD activity and has a temperature sensitive mutation in recA.

32. The method of claim 25, wherein the human gene sequence is selected from the group consisting of genes encoding G-protein coupled receptors, kinases, phosphatases, ion channels, nuclear receptors, oncogenes, cancer suppressor genes, viral receptors, bacterial receptors, P450 genes, insulin receptors immunoglobins metabolic pathway genes, transcription factors, hormone receptors, cytokines, cell signaling pathway genes and cell cycle genes.

33. The method of claim 25, wherein the third DNA construct is a bacterial artificial chromosome.

34. The method of claim 25, wherein the human DNA sequence is a human gene sequence having at least one intron contained therein.

35. The method of claim 25, wherein the third DNA construct has a selection marker contained within the intron.

36. The method of claim 35, wherein the selection marker is added following the recombining step.

37. The method of claim 35, wherein the selection marker is a positive selection marker.

38. The method of claim 35, wherein the third DNA construct has a second selection marker that flanks the non-human animal DNA sequence.

39. The method of claim 25, wherein the human DNA sequence and the first non-human DNA sequence in the second construct are joined to the 5′ of a start codon in a human gene coding sequence.

40. The method of claim 39, wherein the human DNA sequence and the second non-human DNA sequence in the second construct are joined to the 3′ of a stop codon in the human gene coding sequence.

41. A humanized animal produced by the method of claim 1.

42. The humanized animal of claim 41, wherein the animal is a mouse.
